(19) United States
(12) Patent Application Publication
(10) Pub. No.: US 2002/0123671 A1
(43) Pub. Date: Sep. 5, 2002

(54) Method and apparatus for monitoring biological properties

(76) Inventor: Peter D. Haaland, Louisville, CO (US)

Correspondence Address: CHRISTIE, PARKER & HALE, LLP, 350 WEST COLORADO BOULEVARD, SUITE 500, PASADENA, CA, 91105, US

(21) Appl. No.: 09/945,003

(22) Filed: Aug. 31, 2001

Related U.S. Application Data
(60) Provisional application No. 60/230,225, filed on Aug. 31, 2000.

(51) Int. Cl. A61B 5/00

(52) U.S. Cl. 600/300; 600/301

(57) ABSTRACT

A delocalized apparatus for monitoring a biological property in an individual or group of individuals is provided, and includes one or more user terminals having an input port and a user interface, a transducer associated with each user terminal and coupled to the user terminal input port, a controller, and a bidirectional link from the transducer to the controller. A single controller can be used to process data received from multiple transducers, allowing a biological property in a group of individuals to be monitored.

### CROSS REFERENCE TO RELATED APPLICATION

This application claims priority of Provisional U.S. Patent Application No. 60/230,225, filed Aug. 31, 2000, the subject matter and entire contents of which are incorporated by reference herein.

### FIELD OF THE INVENTION

This invention relates generally to the monitoring of biological properties, also known as “biological characteristics” or “biological function” of organisms. Biological properties includes physical, chemical, and biological characteristics of specimens collected from an organism, such as glucose concentration of blood or urine, hormone concentrations in vaginal excretion, sperm concentration in seminal fluid, antibody concentration in saliva, and the like, as well as physiological attributes (i.e., performance or response) of the organism or its systems, such as ophthalmic refractive error, auditory response, pulmonary performance, auscultation (heart and lung) sounds, and the like.

### BACKGROUND OF THE INVENTION

The prior art for monitoring biological functions is extensive but can be divided into two classes. The first class involves trained medical personnel in the collection of samples or raw performance data and its analysis using trained professionals and complex laboratory equipment. The second class involves untrained or minimally trained personnel and simple, inexpensive equipment. The classes are further distinguished by the precision, accuracy, and cost of the data provided, being generally higher for the first than for the second class.

As an example of the first class method, a doctor or nurse draws several milliliters of blood by syringe and sends it to a medical laboratory or hospital where it is analyzed with a clinical blood glucose monitoring system such as the Accu-Chek Advantage GTS (Roche Diagnostice/Boehringer Mannheim), Precision G (Medisense), One Touch II Hospital (Lifescan), Encore QA+ (Bayer), or the like. This first class method provides accurate and precise glucose concentrations but is slow, expensive, and requires trained personnel with complex equipment. A survey of the accuracy and precision of these instrumental methods is disclosed in Precision and Accuracy Evaluation of Four Hospital Blood Glucose Monitoring Systems by S. Jennings, E. Miller, T. Pacheco and M. Brooks (Boehringer-Mannheim technical publication, 1998 available at http://us.labsystems.roche.com/aacc.htm), the entire contents of which is incorporated by reference herein. The second class method for measuring blood glucose concentration involves a patient pricking his or her finger and placing a drop of blood on a strip that contains chemical reagents, such as a mixture of glucose oxidase and ferricyanide, as disclosed in Freitag (U.S. Pat. No. 4,929,545). The reaction of the reagent with glucose produces either a colorimetric (e.g. Galen et al., U.S. Pat. No. 6,027,692) or electrochemical change (e.g. Szuminsky et al., U.S. Pat. No. 5,108,564), which is recorded on a separate, simple meter. All three patents are incorporated by reference herein in their entirety. The resulting measurement has inherently lower precision than the first class method due to uncertainty in the blood volume, the age and chemical activity of the reagent test strip, calibration of the inexpensive photometer or electrometer, confounding reactions with the test reagents, and other such factors. The accuracy and precision of the second class method is disclosed by G. Brunner et al. in Validation of Home Blood Glucose Meters with Respect to Clinical and Analytical Approaches (Diabetes Care, 21 (4), 1998 p 585-90, the entire contents of which is incorporated herein. In particular, Brunner et al. conclude that “[n]one of the devices meet the American Diabetes Association criteria . . . Analytical performance of currently available home blood glucose meters differs substantially within defined glycemic ranges.”

A second example illustrates the two classes of prior art methods of testing for human pregnancy. The first class method involves a blood sample acquired by a nurse or doctor that is sent to a medical laboratory for titration of human chorionic gonadotropin (hCG) or follicle stimulating hormone (FSH). The assay for FSH in blood is complex and involves a solid phase enzyme-linked immunosorbent assay (ELISA). The test requires laboratory personnel, eight different reagents, precision pipettes, and an optical microplate reader as described, for example, in sales literature found at the web-site of KMI Diagnostics, (http://www.kmidiagnostics.com/FSH.htm.) The second class method uses a qualitative color change to a urine-soaked test strip that is treated with a chemical reagent that reacts with hCG. Commercial examples include the Fact Plus product, made by Ortho Pharmaceutical Corp. Raritan N.J. 08869, and the Clear Blue Easy product, manufactured by Whitehall Laboratories, Madison N.J. 07940. The first class method is slower, more expensive, and quantitatively precise, producing a numerical concentration of the hormone and a confidence interval or variance. In contrast, the second-class method is quick, inexpensive, and less precise, producing a yes-or-no indication of pregnancy based on an arbitrary threshold hCG concentration.

A third example of the two diagnostic classes involves characterization of nevi (moles) in human skin. The current screening methods for pre-cancerous or malignant moles require a physicians' qualitative examination of the mole shape, size, and color followed by tissue excision and laboratory biopsy to confirm the physicians' diagnosis. This procedure is expensive and requires trained pathologists to evaluate the malignancy of the excised tissue by microscopic examination. The second class method involves visual inspection and the ‘ABCD’ rule, where self examination for Asymmetry, Border irregularity, Color, and Diameter is used to screen for potentially malignant moles as described, for example, at the web site http://ww.eurohealth. ie/cancom/skin07.html. This method is simple and inexpensive, but not very accurate. See Whited and Grichnik, Journal of the American Medical Association, 279, 696-701 (1998) the entire contents of which is incorporated by reference herein.

The previous examples illustrate two classes of monitoring biological properties using data from a specimen collected from an organism. The monitoring of biological properties using a measurement or evaluation of a physiological attribute is similarly exemplified for two classes, as described below.

Human pulmonary function is governed by the performance of the muscular, skeletal, and nervous systems, and also by the fluid mechanical properties of nasal, esophageal, and lung tissues. Characterization of pulmonary function is accomplished using the first class method by a physician, nurse, or respiratory therapist recording the volume of air inhaled and exhaled by a patient into a spirometer, such as the Spirotek SP-1 manufactured by Welch-Allyn (Skaneateles, N.Y.), in a clinical setting. The second class method is exemplified by a simple ballistic exhalation meter that is prescribed for use by asthma patients. This device measures the peak force or flux of exhaled air by recording deflection of a spring, which is then recorded on a chart or graph by a patient. This ballistic exhalation test requires minimal training and inexpensive equipment but is substantially less accurate and contains less information than in-office spirometry methods.

Another example of monitoring biological properties based on a physiological attribute of the organism is the measurement of heart and lung sounds, also known as auscultation. The first class method involves a nurse or physician manually placing a stethoscope at varied locations on the chest and listening to hear qualitative sounds of murmers, lung congestion, and other pathologies of the heart and lungs. The art of auscultation is thoroughly described by E. Stein and A. Delman in Rapid Interpretation of Heart Sounds and Murmurs, 4^{th} Edition (Lippincott Williams & Wilkins, 1996), the entire contents of which is incorporated by reference herein. The sophisticated training required to diagnose heart and lung sounds, as well as the finesse required to orient the stethoscope to minimize noise and confounding artifacts, have heretofore precluded a second class method for monitoring auscultation, although inexpensive probes of pulse are available to monitor heart rate only.

A third example of monitoring a physiological attribute of an organism is the measurement of refractive error in human eyes. Refractive error in the eye is well known and is currently characterized by sophisticated measurements performed by an optometrist, ophthalmologist, or other trained professional using a suite of precise corrective lenses and an eye chart in order to prescribe corrective spectacles, contact lenses, or laser surgery. This measurement requires an array of expensive optics and interaction with a trained clinician to extract the patients' corrective prescription. The second class of refractive error measurement involves only an eye chart with no optics, as commonly employed by state governments for the issuance of driver's licenses. This method can be used to screen for refractive deficiencies but not to identify the magnitude of spherical or cylindrical error, or the axial orientation of astigmatism, that are required to optimize visual acuity. The first class method is distinguished by complex equipment, highly trained personnel, and precise, accurate measurements, while the second-class method is characterized by simple equipment, marginally trained personnel, and results that have degraded precision and accuracy.

The above examples illustrate the dichotomy of the prior art for characterization of biological function into two classes that correlate the precision and accuracy of the diagnostic data with the complexity of the measurement, the training of the user, and the overall cost of the measurement. In other words, the prior art achieves accuracy and precision at the expense of measurement complexity and the need for trained personnel to sample and analyze results. Additional examples will be familiar to those practiced in the art of monitoring biological specimens and attributes of biological systems.

In view of the limitations of the prior art, there is a need for improved methods and apparatus for monitoring biological properties of organisms, especially of humans. In one sense, the problem is to provide the improved accuracy and precision of the first class diagnostic methods while using only the marginally trained practitioner or layperson and simple equipment of the second-class method. A further problem with the prior art is that accurate diagnostic procedures are expensive and often logistically inconvenient, requiring office visits, shipment of specimens, and delays while samples are analyzed. A related problem with prior art methods is that their expense and the inconvenience of repetitive measurements at regular intervals precludes establishment of baseline conditions for individual clients. Pathological conditions are clinically indicated when diagnostic values such as glucose or hormone concentrations, ophthalmic prescription, heart murmers, and the like exceed average values for a large population rather than a measurable change in an individual with time. In other words, the implicit assumption of prior art diagnostic methods is that time averages and ensemble (population) averages are statistically equivalent. A need exists for a method and apparatus that overcomes these problems.

### SUMMARY OF THE INVENTION

In accordance with the present invention, a method and apparatus for monitoring a biological property in an individual or group of individuals are provided. In an exemplary embodiment, one such apparatus includes at least the following components: a user terminal, having an input port and a user interface; a transducer coupled to the input port; a controller; and a bidirectional link from the transducer to the controller. Advantageously, the user terminal and controller have geographically distinct locations. The transducer is capable of converting a biological input collected at the user terminal into a first signal; the controller is capable of processing the first signal, generating a second signal, and causing the second signal to be transmitted over the bidirectional link to the user terminal; and the user terminal is capable of converting the second signal into a human-discernible message.

A method for monitoring a biological property, according to one embodiment of the invention, makes use of these components, and comprises the steps of collecting a biological input at the user terminal; converting the biological input into a first signal in a transducer associated with the user terminal; transmitting the first signal over a bidirectional link to a controller; processing the first signal in the controller and generating a second signal; transmitting the second signal over the bidirectional link to the user terminal; and converting the second signal into a human-discernible message at the user terminal.

In accordance with another embodiment of the invention, a system for monitoring a biological property in a group of individuals is provided, and includes at least one controller; a plurality of user terminals, each having an input port and a user interface; a plurality of transducers, with each transducer coupled to the input port of one of the user terminals; and a plurality of bidirectional links, with each link “coupling” a transducer to the controller. With such a system, a method for monitoring biological properties in a group of individuals is made possible, and represents another aspect of the present invention. According to one embodiment of the invention, the method includes the steps of collecting a biological input at each of the plurality of user terminals; converting each biological input into a first signal in a unique transducer associated with each of the user terminals; transmitting each first signal over a unique bidirectional link to the controller; processing all of the first signals in the controller and generating a plurality of second signals; transmitting a second signal over each unique bidirectional link to each user terminal; and converting each second signal into a human-discernible message at each user terminal.

An advantage of the present invention is that it provides a delocalized diagnostic method; the controller, transducer(s), and electromagnetic link are in different locations. A further feature of the present method is that feedback between the controller and transducer following analysis of the first data stream permits improved accuracy and precision of the quantitative measurement of biological specimens through statistics, error checking, and numerical modeling of expected results. Yet another feature of the present invention is the ability of the controller to intercompare raw data from a plurality of identical transducers being used by different individuals. In other words, the invention enables population-based health management. As will be clear to those practiced in the art of analytical chemistry and statistics, this method permits both quantitative values and confidence limits to be provided for biological specimens or physiological attributes collected or monitored by minimally trained observers using inexpensive transducers at remote sites.

### BRIEF DESCRIPTION OF THE DRAWINGS

FIG. 1 is a schematic illustration of an apparatus according to one embodiment of the invention;

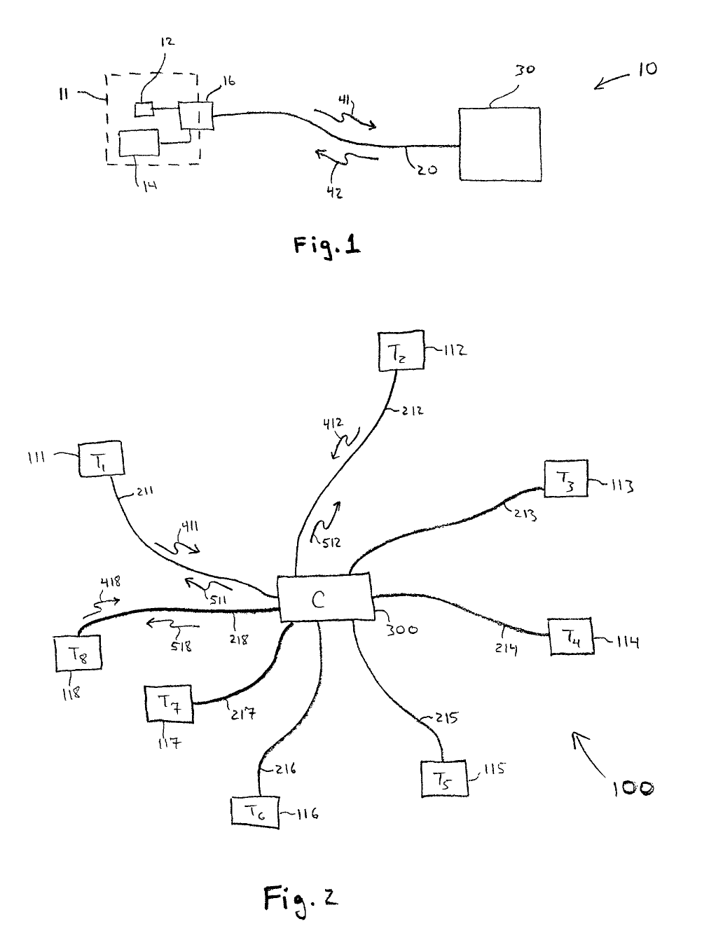

FIG. 2 is a schematic illustration of an apparatus according to a second embodiment of the invention;

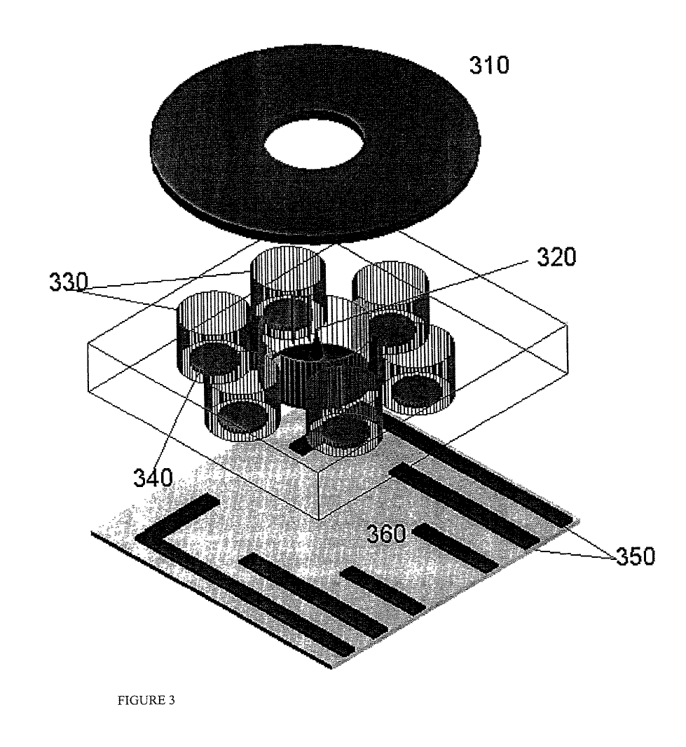

FIG. 3 is an exploded view of a test strip for electrochemical analysis of glucose concentration in blood, according to one embodiment of the present invention. A conducting reference electrode (310) is perforated to allow puncture of skin by lancet (320) and flow of blood into the six electrochemical cavities (330). An electrical potential is applied between (310) and the sampling electrodes (340) using conducting strips (350) that are connected to the terminal with a sliding electrical connection at the edge of the insulating plane (360);

FIG. 4 is a schematic view of a wedge-shaped specimen cell and transducer for calorimetric analysis of blood, urine, and other fluid specimens, according to one embodiment of the present invention. Biological fluid such as blood, urine, saliva, tears, or the like are admitted to sample volume 410 through an aperture 420 that is mated to a syringe or dropper. A suitable air vent such as a pinhole is provided in the top or bottom face of the device (not shown). The fluid reacts with reagents that are contained in the volume or on the surfaces of the cavity 410. Light is generated from source 430, which may be light emitting diodes, an electroluminescent strip, an incandescent lamp, or the like. The light is scattered, transmitted, and absorbed as it passes through the sample in cell 410, after which it is spectrally filtered through an absorbing or reflective filter 440. The resulting light is imaged by a microlens array 450 onto a photodiode array or other spatially resolved light sensitive device 460, from which signals are sent to the controller;

FIG. 5 is a graph showing typical concentrations of several hormones following conception, and is abstracted from Guyton and Hall (op cit.). Secretion rates of human chorionic gonadotropin (hCG), estrogens, and progesterone through the course of normal human pregnancy. (Guyton and Hall, Textbook of Medical Physiology, (Philadelphia:Saunders) 1996, p 1037);

FIG. 6 is an exploded view of a sample cell used for evaluation of cultured samples according to one embodiment of the present invention, having a geometry that permits simultaneous heating, cooling, and optical observation of the cultured specimens. Transparent windows 610 and 640 are bonded to the cell matrix 625. Biological fluid is added through an aperture in window 610 and is diverted into each of the culture cells 625 by deflection from the hydrophobic dimple 630. After addition of the sample the six cell block is clamped between an electrically controlled heating element 660 and an electrically controlled cooling element 650 so that heat transfer to and temperature of the culture media are controlled. The temperature control elements 660 and 650 have apertures that allow unobstructed optical interrogation of the culture media.;

FIG. 7 is an exploded, schematic view of a transducer and test strip for genetic (PCR) analysis according to one embodiment of the present invention. The test strip contains a reaction cavity that can be thermally cycled and a separation medium across which a the transducer applies a controlled electrical potential so that reaction products may be separated based on their mobility in the applied electric field and detected by absorption of optical radiation as they move along the separation medium. Analyte is added to reaction chamber 710 and thermally cycled through the PCR steps. At the end of the cycle a potential is applied between end cap electrodes 760 and porous grid electrode 720 in order to concentrate the analyte near grid 720. Under direction of the controller grid a potential is then applied between electrodes 760 and grid electrode 720 is set to an intermediate potential consistent with electrophoretic transport of the analyte through gel medium 730. The analyte separates into bands of different electrophoretic mobility that are detected in real time by passing light from source 740 through gel medium 730 and onto the optical detector array 750;

FIG. 8 is a collection of graphs showing typical waveforms for mass and volume flow, pressure, and instantaneous volume from which pulmonary function metrics are computed by the controller according to one embodiment of the invention. Idealized volume flow rate (cm³/s), mass flow rate (g/s), pressure (cm H₂O), and volume for inhalation from time t=0=>6.2 and exhalation from t=6.2=>12.4 seconds; and

FIG. 9 is a schematic illustration arrangement in 3-dimensional (upper) and cross-sectional (lower) aspects of a transducer for sensing intraocular pressure using a piezoelectric speaker to transmit acoustic waves to the eyeball and an optical cantilever to monitor the mechanical deflection of the cornea in response to the acoustic pressure, according to one embodiment of the invention. Eyecup 920 has an integrated piezoelectric speaker or pulsed gas valve 970 that is placed over the eye 910. An eye-safe probe laser 950 directs a beam 930 that is reflected from the corneal surface of the eye 910 and imaged onto a position sensitive light detector 960. The motion of the cornea in response to acoustic excitation is recorded as a time dependent deflection of the optical cantilever comprised of 930,940,950, and 960.

### DETAILED DESCRIPTION OF THE INVENTION

According to the present invention, a delocalized method and apparatus for monitoring health, disease and, more generally, biological properties, in an individual or group of individuals is provided. In one embodiment, an apparatus includes a user terminal, having an input port and a user interface; a transducer coupled to the input port; a controller; and a bidirectional link from the transducer to the controller. The transducer and controller are “coupled” to each other by the bidirectional link, but not necessarily physically connected. Preferably, the user terminal (and its associated transducer) are in a location geographically distinct from the controller, making the apparatus “delocalized” and providing the attendant advantages described herein.

In one embodiment, the user terminal comprises a personal computer (PC), and the user interface includes a keypad, mouse (or other cursor control device, such as a TouchPad), a monitor, and optionally, a microphone and/or speaker(s). In another embodiment, the user terminal comprises a dedicated, microprocessor-controlled station other than a PC, and the user interface includes one or more of a switch, pushbutton, rheostat, keypad, or other means for entering data into and/or prompting the microprocessor. The user interface may also include a video monitor, microphone, speaker(s), etc.

The input port is configured to receive a biological specimen and/or a physiological signal, image, or response. Non-limiting examples include sample cells and other receptacles and means for receiving specimens and/or physiological input as described herein and generally known to persons skilled in the art.

In general, the transducer, the input port, and/or the combination of the two is a relatively simple and inexpensive device that is used by an untrained or minimally trained person to collect specimens or measure a physiological attribute of a biological organism or system. The controller is a relatively complex and expensive device (e.g., a computer server or cluster of digital workstations) that processes signals from the user terminal(s) and thereby characterizes or programs the state of the transducer(s), triggers the transducer(s)' operation, interprets data contained within signals from the transducer(s), evaluates the precision and accuracy of the raw data, and prompts the transducer(s) for additional measurements or data as required to achieve a desired precision and accuracy of the method. The bidirectional link allows signals to flow between the transducer(s) and the controller. Non-limiting examples include links capable of carrying electric, magnetic, optical, and other electromagnetic radiation, etc. More specific (but also non-limiting) examples include telephone lines, coaxial cables (such as found in cable television connections), satellite transceiver connections (line-of-sight or otherwise), the Internet, microwave, radio, infrared, and other frequency electromagnetic radiation transmitter/receiver connections, fiber optic connections, and similar links familiar to those practiced in the art of communication.

In practice, a plurality of signals are transmitted back and forth from the transducer(s) and controller. A user may, for example, turn on his or her PC, initiate a program relating to the biological property to be monitored, and follow on-screen prompts and other instructions, as described below.

There are several aspects to the invention. The following non-limiting examples are provided to illustrate the method of delocalized biological diagnostics as disclosed herein.

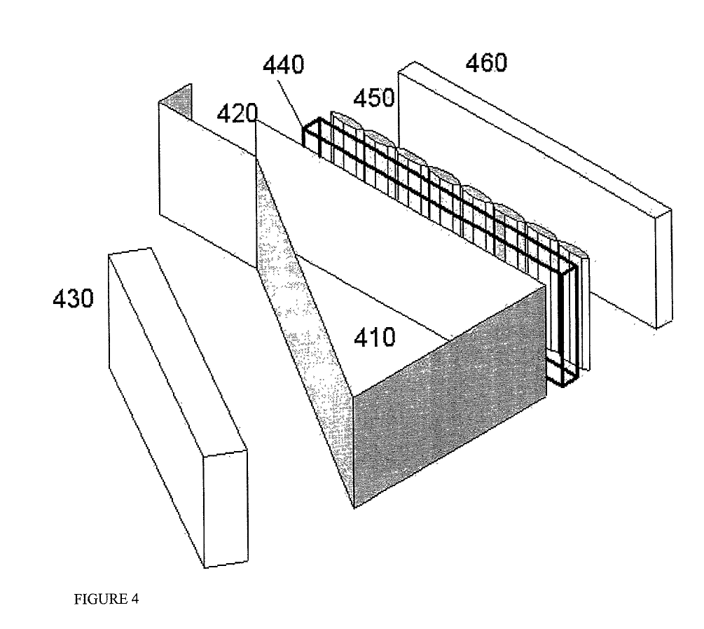

Glucose monitor: The concentration of glucose in blood and interstitial fluids is an important quantity for the detection and treatment of diabetes. According to one embodiment of the invention a transducer is comprised of an electrical device that measures current as a function of applied voltage. Disposable test strips contain a plurality of electrochemical cells that are filled with blood by capillary action as shown in FIG. 3. The cells are micro-machined from silicon wafers or lithographically patterned from polymeric substrates, using standard etching and deposition methods familiar to those practiced in the art of microelectronic fabrication, to give a plurality of precisely defined volumes and electrode areas. The surfaces of the entrance port to the cell are preferably coated with materials that enhance capillary action by changing the contact force on a drop of blood. The sample cavity is coated with hydrophobic polymer, while the channels and sample cells are coated with hydrophilic polymer. The contact force is a result of balance between surface tension of the liquid, gas (air), and solid surfaces. The amplitude of this force acting on a fluid specimen can be controlled by adjusting the composition of the surface or by dissolving surfactant materials in the fluid, as is well known to those practiced in the art of rheology. A sample of blood is applied to the center well of the test strip and responds to the contact force by flowing into the electrochemical cells, which have been pretreated with calibrated quantities of chemical reagents that react with glucose to produce a calorimetric shift as described in U.S. Pat. No. 5,462,064 to D'Angelo et al. or an amperometric (electrical current) signal as set forth in U.S. Pat. No. 5,108,564 to Szuminsky et al., both references being incorporated herein. The reagents generally include an enzymatic material that selectively reacts with glucose, such as glucose peroxidase or dehydrogenase, and reagents that indicate the products of its reaction with glucose by color or oxidative changes.

The test strip has a serial number that is read by the transducer, for example as a barcode printed on the back surface of the strip. The serial number is transmitted to the controller, which then verifies the date the strip was prepared and the manufacturing lot numbers for the reagents and device. As blood flows into each of the cavities, their electrical impedance (capacitance) is shifted. The controller, which monitors the electrical impedance of the cells, verifies the status (empty, partially filled, filled) of each cell and triggers the beginning of one or more electrochemical measurements when the cell is full of specimen. A voltage waveform V(t) is applied to the electrodes and the corresponding current waveform, I(t), is recorded by the transducer and communicated through the link to the controller.

The bidirectional link between the controller and transducer may comprise any of a variety of electromagnetic means, a non-limiting list of which includes a cellular phone link, a telephone line, a wireless world-wide-web link through a Palm pilot or other personal digital assistant, a serial port into a computer that is in turn connected to the internet or a local area network (LAN), an infrared data link to a computer that is in turn connected to the internet or a LAN, a cable television line, and a radiofrequency link such as is used to activate personal paging devices.

The controller is preferably a server or a cluster of digital computer workstations, and is linked to the transducer(s). The controller provides instructions to initiate measurements, and reads raw data that is transmitted from the transducer(s). Preferably the controller's computers are linked by a scalable, concurrent, computing architecture so that the storage and processing capacity of the controller are efficiently matched to a desired number of transducers. The controller uses commercially available software such as OpenDB (Oracle, Burlingame, Calif.) for database administration, PGP (Network Associates, Palo Alto, Calif.) for client information security, Matlab (The Math Works, Natick, Mass.) and Mathematica (Wolfram Research, Urbana, Ill.) for signal processing and statistical analysis, Java (Sun Microsystems, Santa Clara, Calif.) for internet linking, and the like. The controller also preferably uses customized algorithms for each diagnostic challenge based on the statistical and mathematical properties of the transducer signals and the spectrum of possible diagnostic outcomes.

The voltage and current waveforms are mathematically converted by the controller into cyclic voltammagrams (I(V) curves) and other mathematical transforms of the raw data such as peak current, integrated charge, separation of peak oxidative and reductive potentials, and the like in order to quantify the concentration of glucose and its uncertainty or variability. In one embodiment, the sample cells have the same volume, electrode geometry, and reagent (enzyme) concentrations. If the results from each identical cell are equal within preselected quality standards then they are displayed and archived by the controller. These preselected quality standards are primarily derived from clinically acceptable accuracy and precision as set forth, for example, by the American Diabetes Association as described in Brunner et al (loc. Cit.) and also at the ADA web site http://www.ada.org, the entire contents of which is incorporated by reference herein, and secondarily by the instrumental characteristics of the transducer. If the variability of results among the sample cells is higher than the predetermined standards, algorithms are applied by the controller, both to the raw data and to characterization of the transducer or test strip, to identify the source of variance. The controller processes this data and initiates reacquisition of waveforms or prompts the patient to repeat the test with different reagents or conditions to obviate the variability.

In a preferred embodiment, the cells have different volumes, electrode areas, and reagent concentrations that are chosen to give reliable measurements over the widest possible range of expected values. The raw data are analyzed and compared to check whether scaling relationships such as peak current versus electrode area, total charge versus total volume of blood, rate of change of current versus reagent concentration, and the like are satisfied for the sample. If the data are consistent within a predetermined margin of error they are displayed and archived by the controller, otherwise an algorithm to identify the source of the uncertainty and/or prompt for additional measurements is invoked.

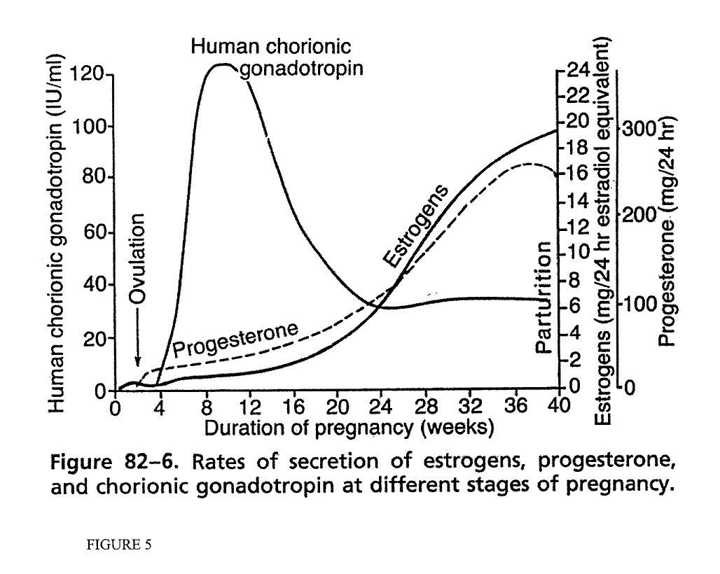

In another preferred embodiment of the invention, the glucose concentration measurements are made on the same sample by qualitatively different analytical techniques. A test strip according to this embodiment includes a wedge-shaped cavity bounded by transparent walls that have been pretreated with enzyme and indicator dye, as shown schematically in FIG. 4. The wedge-shaped test strip is inserted into a transducer that includes a light source that illuminates the wedge and a plurality of light sensors that record the intensities of transmitted light at several positions. Light incident on the wedge is scattered and absorbed by the specimen, then focused by lenses, detected by photodiodes, and transmitted to the controller. The light passing through the cell may be reflected, scattered, transmitted, or absorbed. Each photodiode samples the transmitted and scattered light through a different, known volume of fluid at a series of times according to the optical characteristics of the cell, windows, lenses, light source, and scattering by optical inhomogeneities in the sample. The absorption of light varies with both time and position according to formulae that are well known to those practiced in the art of physical chemistry and optics. The absorption of light by analyte (i.e. the specimen component being diagnosed) is logarithmically proportional to the product of analyte concentration and path length (Beer's law), while the rate of analyte production is proportional to the product of analyte and reagent concentrations and varies exponentially with the temperature (second-order chemical kinetics). The controller mathematically inverts the equations of optical absorption and chemical kinetics using the time-dependent photodiode signals transmitted from the transducer to provide measures of the glucose concentration and its uncertainty. If the uncertainty is within acceptable bounds then the measurement is displayed and archived; otherwise the controller initiates an algorithm to identify the noise or error and prompts for additional measurements are initiated.

In yet another embodiment of this aspect of the invention (blood glucose monitoring), the electrochemical and photochemical methods described above are combined using a single transducer-strip combination. The controller compares the glucose concentrations inferred from electrochemical and photometric measurements and, as before, displays and archives data that are within an acceptable margin of error; otherwise the controller initiates an error tracking algorithm and prompts for additional measurements.

Another embodiment of the glucose monitoring aspect of the invention allows separate determination of the glucose bound to protein (Hemoglobin) and freely dissolved in blood plasma. Glucosylated hemoglobin is well known to be an indicator of blood glucose concentrations averaged over periods of one to three months and thus has diagnostic value for the treatment of diabetes mellitus, as set forth in U.S. Pat. No. 6,027,692 to Galen et al., the entire contents of which is incorporated herein by reference. In this embodiment, one or more of the channels connecting the sample inlet to electrochemical (FIG. 3) or photochemical (FIG. 4) cavities is occluded by material that impedes protein transport. This material may be a filter, membrane, molecular sieve, chemically activated surface, or other medium that selectively binds or obstructs the flow of hemoglobin. Alternatively, the channel shape, size, surface texture, and surface composition may be selected to allow plasma flow but not hemoglobin (protein) flow. Comparison of the electrochemical assay for cells with and without hemoglobin are then performed as described herein to permit partitioning of the blood glucose concentration between free and protein-bound forms. As described previously, the accuracy and precision of the data are compared and archived if they satisfy clinically acceptable values; otherwise the controller initiates error checking algorithms and prompts for new samples.

In summary, one embodiment of a method for glucose monitoring according to the present invention comprises the following steps:

1. A client applies a specimen to a test strip that has a plurality of reservoirs;

2. The client inserts the test strip into a transducer that is linked to a controller by electromagnetic means;

3. The controller verifies the lot numbers of the test strip and transducer and the presence of fluid in each cavity;

4. The controller triggers a series of electrical or optical measurements by the transducer in each cavity;

5. The transducer relays raw data over the link to the controller;

6. The controller analyzes the raw data and compares results for different cavities based on mathematical models of cell performance and statistical variability; and

7. The controller calculates the blood glucose concentration and its variance among the plurality of measurements from each cavity. Results with variance below a preselected threshold are reported to the client over the link and archived by the controller in a database. Results with excessive variance are analyzed further by the controller, which then prompts the transducer to perform additional measurements or the user to repeat the test with a different test strip.

It will be appreciated that the steps described above (as well as the general approach described in other examples and passages herein) can be readily carried out with other blood analytes, as well as with other bodily fluids and various analytes contained therein.

Feedback between the controller and transducer produces improved accuracy and precision when compared with stand-alone or telemedicine methods. For example, a stale or otherwise impure lot of reagent will be detected by the controller the first time that it is used. Subsequent measurements by other transducers at diverse locations with the same reagent lot will be adjusted or cancelled by the controller. The accuracy of the method is further improved by severely limiting the variability caused by subjective client interpretation; for example, a precise volume rather than a drop of blood is used for evaluating the glucose concentration. Accuracy and precision are also increased by the statistical analysis of very many measurements made using disparate transducers with the same controller-driven algorithms and methods.

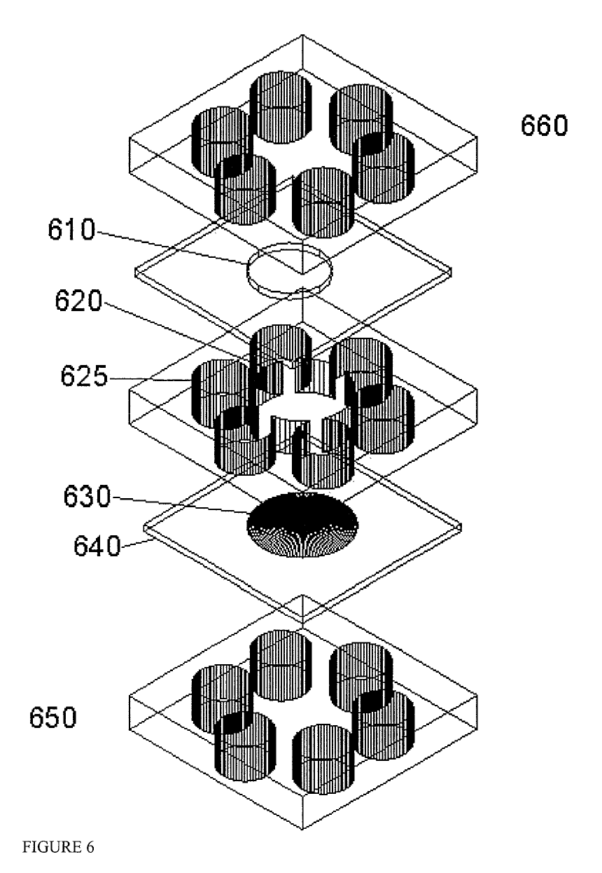

Pregnancy Test: A second aspect of the method according to the present invention involves biological specimens other than blood and allows determination of human pregnancy. Pregnancy tests that monitor human chorionic gonadotropin (hCG) are well known in the art. The hormone hCG is produced following conception and is essential to normal pregnancy. The production of hCG and its concentration in blood plasma and urine typically varies during pregnancy as shown in FIG. 5.

A description of hormonal shifts during pregnancy is described by Guyton and Hall, Textbook of Medical Physiology, (Philadelphia:Saunders) 1996, chapter 82, which is incorporated by reference herein. According to the present invention, a sample of urine is drawn into a simple plastic syringe (with no needle) and supplied to a wedge-shaped photochemical cell (FIG. 4) that has been pretreated with chemical reagents that change color when they react with hCG. The cell is connected to a transducer so that light produced by a source is transmitted through an optical filter and the cell prior to being imaged by microlenses onto a photodiode array as shown in FIG. 4. One or more than one cell may be used at each transducer, with each cell having different volume, geometry, or calorimetric reagents. Urine is supplied to the micromachined cell(s), which are illuminated by a light source (light emitting diodes, filtered incandescent lamps, or the like) emitting light with predetermined wavelengths. Depression of the syringe fills the cells as indicated by a change in their optical transmission. (Note that the transmission through the cell is reduced by Fresnel reflection from the internal cell faces when the cavity is filled with air. The introduction of fluid decreases the refractive index contrast at the cavity boundary and thereby the Fresnel loss at these cell faces, so that addition of fluid increases the quantity of transmitted light in the absence of absorption.) The controller senses this change and starts a timer to record the time-dependent optical signal along a plurality of optical paths in the test strip. The optical paths traversed by light from the source through the cell, fluid, filter, and microlens to the detector element differ for each detector element in a manner that is prescribed by geometrical optics, and as such is familiar to those practiced in the art of optical design. As described above, both the peak filtered light intensity and the time dependence of the transmitted light's evolution are recorded by the controller. The controller then computes the hCG concentration independently from the rate of color change and its peak intensity. If the values agree within required tolerances, then a result is archived and displayed by the controller; otherwise the controller initiates algorithms to check for sources of error and prompts for additional measurements. One such error may be caused by the presence of small particulate matter such as granulocytes (white blood cells) in the sample that scatter rather than absorb light. The controller uses the measurements at multiple paths and wavelengths to indicate and correct for attenuation changes due to scattering and thereby improve the reliability of the hCG concentration. According to the present invention, measurements at more than one date permit the controller to compile a time series of accurate specimen properties such as hCG concentration, estrogen concentration, and the like. Prior art methods of home pregnancy testing such as the AimStick Pregnancy Test (Germain Laboratories, Inc. San Antonio, Tex. 78229 (detects threshold level of 20 mIU of hCG), the Clearblue Easy (Whitehall Laboratories Madison N.J. 07940 (detects threshold 50 mIUs of hCG)) and the like rely on a single threshold concentration measurement. In other words, when the hCG level shown in FIG. 5 rises above this threshold a ‘positive’ test results. There is no indication of the precise concentration, the term of pregnancy, or the extent to which the measured value exceeds the threshold. The method described herein permits evaluation of the actual hCG concentration and its derivative with respect to time. This derivative may be used, for example, to estimate the elapsed time from conception to measurement and thereby the term of the pregnancy.

A second embodiment of this aspect of the invention employs a transducer with a plurality of cavities that draw from the same specimen, as would be obtained from a stack of the test strips displayed in FIG. 4. Each cavity is treated with different chemical reagents to simultaneously evaluate the concentration of hormones such as human somatomammotropin, as described in U.S. Pat. No. 4219467 to Pende et al. and incorporated herein; fibronectin, as set forth in U.S. Pat. No. 5,281,522 to Senyei et al. and incorporated herein; or other proteins such as estrogens or progesterone whose concentrations are correlated with the clinical state of pregnancy.

From the foregoing it can be seen that the present invention improves the accuracy and precision with which hormone concentrations are determined by minimally trained personnel with simple equipment. The improved accuracy and precision are obtained by

1. reducing measurement variability using the controller to automate the complex facets of signal acquisition and analysis;

2. quantifying variability by replicate measurements under the same or systematically altered reaction conditions; and

3. archiving results so that variability and precision among samples acquired by diverse clients in disparate locations can be analyzed and quantified.

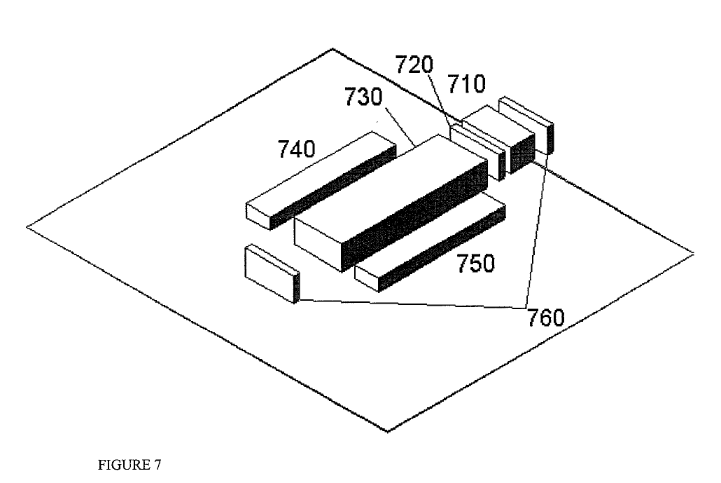

Bacterial cultures: A third aspect of the present invention facilitates accurate analysis of bacterial and other cultures. The transducer used in one embodiment of this aspect of the invention is a microfluidic cell with top and bottom faces that are optically transparent and thermally conductive. Fluid specimens such as blood, tears, semen, saliva, throat swab extract, urine, sputum, mucous, or the like are introduced to the sample inlet at the center of a cell array. The fluid is conducted through treated capillary ports to a plurality of sample reservoirs whose volume and chemical contents are preselected for the desired cultures. The sample cell is then clamped into a temperature-cycling transducer such as that shown in the exploded projection of FIG. 6. A thermoelectric cooler and resistive heater are in thermal contact with opposite faces of the sample cell. The temperature of the cell is cycled by the controller to precisely govern the time during which the cells are maintained at temperatures selected to optimize growth of the culture. The optical transmission of spectrally filtered light through each cavity is monitored by detectors at intervals specified by the controller.

In a non-limiting embodiment of this aspect of the invention, thermostatically controlled temperatures are used to optimize the culture of, e.g. Group A beta-hemolytic streptococcus, the infectious agent responsible for strep throat. Culture media are preloaded into the cavities of the sample cell shown in FIG. 4 and clamped between the transducer faces which serve to control the temperature of the sample cell while simultaneously evaluating light transmission through the cavities. Each cavity preferably contains different culture media or different concentrations of a single culture medium. The growth of bacteria causes changes in the absorption and scattering of light within the sample cavities. These time-dependent optical changes are monitored using photosensors in the transducer and evaluated by the controller according to the present method for accuracy and precision using feedback and statistical analysis.

It will be apparent to those practiced in the art of microbiology that the speed and accuracy of results from cultures grown according to the present method will be improved by the replication implicit in the multi-cavity sample cell and the observation of dynamic culture growth in multiple media. It will also be apparent that the method can be practiced by a marginally trained client or practitioner, whose only requirement is to apply a swab or tissue sample to the transducer, since the complexity required to culture the media and periodically read the colony growth is accomplished with direction from and analysis by the controller.

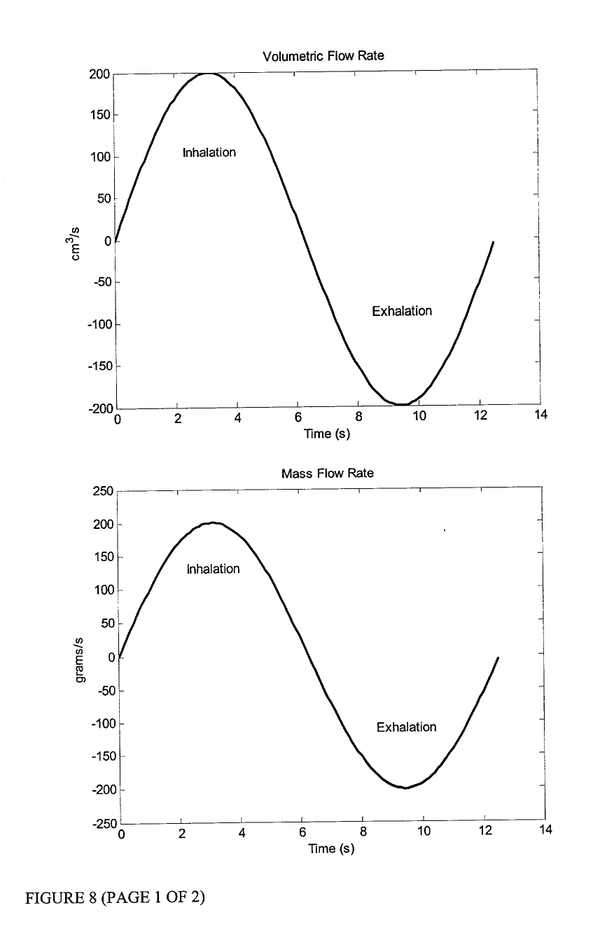

In another embodiment of this aspect of the invention, a test cell such as that shown in FIG. 7 is used to perform polymerase chain reactions (PCR) for amplification of genetic material. The polymerase chain reaction uses repetitive cycling of a mixture of a synthetic oligonucleotide primer pair flanking the DNA sequence to be amplified, a cocktail of dNTPs (dGTP, dATP, dTTP, and dCTP), and Taq DNA Polymerase (AmpliTaq TM, Perkin-Elmer/Cetus), in a sequence of three steps:

I. Denature 93-94 degrees C. 1.5 minutes

II. Anneal 50-65 degrees C. 2 minutes

III. Polymerize 72 degrees C. 2 minutes

As set forth, for example, by H. A. Erlich in PCR Technology: Principles and Applications for DNA Amplification (Stockton Press, New York. 1989), the entire contents of which is incorporated by reference herein. The three step sequence is a typical protocol for PCR amplification. The number of steps, composition of reagents, times, and temperatures may be altered according to protocols that are familiar to those practiced in the art of genetic engineering.

Subsequent to this repetitive cycling as prescribed by the controller, the transducer initiates a separation algorithm that applies an electrostatic field to each cavity so that the components may be spatially separated by their differential mobility in a porous or gel medium. The motion of each component across the gap is recorded when light passing through a specific volume of gel is scattered and/or absorbed. The controller then converts the time-dependent optical attenuation to a chromatographic band structure for comparison with target genetic sequences. The controller analyzes the band structure of each cavity for completeness and consistency before pronouncing a diagnosis to the client.

In other words, in one embodiment of this aspect of the invention, cultured biological specimens are analyzed using the following steps:

1. The client applies a specimen to a test strip and inserts it into a transducer;

2. The insertion of the test strip triggers communication by electromagnetic means with the controller;

3. The controller programs a sequence of heating, cooling, and signal sampling events to be executed by the transducer;

4. The transducer relays signals to the controller for analysis;

5. The controller analyzes the time dependence and statistical variation of data using models of ideal transducer performance in order to quantify the accuracy and variability of the diagnostic result; and

6. Clinically acceptable results are archived by the controller and displayed to the client; unacceptable results are analyzed further and the controller performs additional measurements or requests new samples or transducer configurations.

Thus the present method reduces the variability, labor, and expense associated with human operation of the transducer by providing reproducible, automated, and economical programming afforded from the controller. The invention improves accuracy by replacing the single visual inspection of a cell culture by a laboratory technician with an automated, time-dependent scattering or absorption measurement.

Pulmonary function: In addition to providing new methods and apparatus for characterizing the chemical and biological properties of specimens collected from people (and other biological organisms), the invention also provides new methods and apparatus for characterizing the properties of biological systems, for example, pulmonary function. Pulmonary function depends on the aggregate interaction of muscles, skeletal components, membranes, and orifices as set forth, for example, by J. A. Seikel, D. W. King, and D. G. Drumright, Anatomy and Physiology for Speech, Language, and Hearing, (San Diego: Singular Publishing Group) 1997, especially chapters 3 through 8, which are incorporated by reference herein, and by A. C. Guyton and J. E. Hall, Textbook of Medical Physiology, (Philadelphia:Saunders) 9^{th} edition 1996, chapter 37, which also is incorporated by reference herein. In one embodiment, the transducer is a device that produces an electrical signal that simultaneously determines the pressure and the mass flow rate or volume flow rate of air. The pressure, mass flow rate, and volume flow rate may be determined by a wide variety of means familiar to those practiced in the art of fluid mechanics and described, for example, in Handbook of Transducers by H. N. Norton (New Jersey: Prentice Hall 1989) especially chapters 12 and 15 which are incorporated by reference herein. Non-limiting examples of pressure sensors include thermocouple gauges, capacitance manometers, cantilevered and piezoelectric force sensors. Non-limiting examples of flow sensors include bladed propellers whose rotation is sensed by optical reflection or magnetic induction, and devices that quantify convective cooling of a heated element. The transducer according to one embodiment of the present invention contains at least one pressure sensor and at least one flow sensor to quantify the pulmonary function.

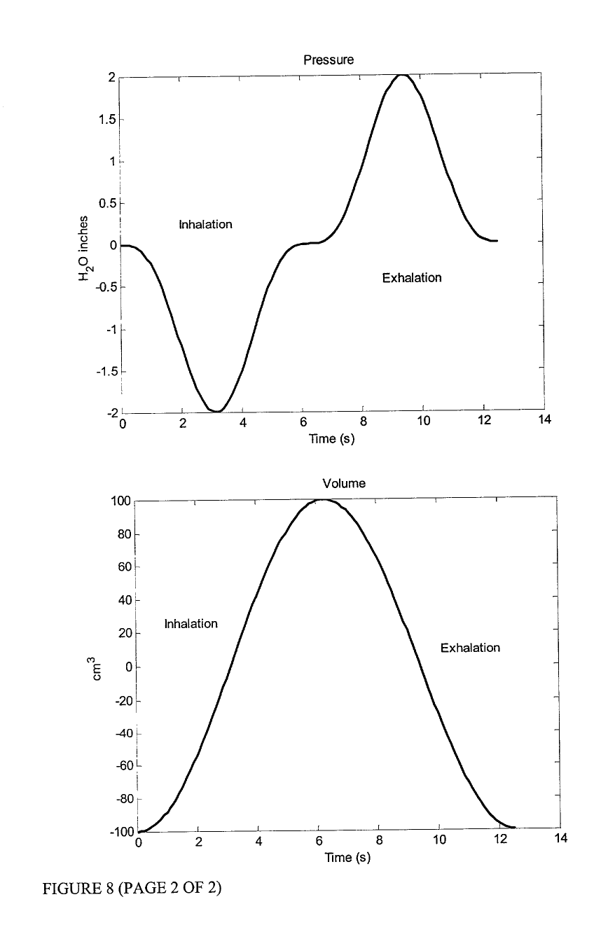

The controller transmits and displays printed or audible instructions to the user through the electromagnetic link. These instructions ask the client to perform a relaxed breath, inhale deeply, pant, cough, and so forth as would a respiratory therapist during an examination. The transducer then relays the time dependent intraoral pressure and volume or mass flow rate of gas through the transducer and computes figures of merit for pulmonary function. The controller performs checks on the data for internal consistency. Idealized representations from a typical measurement of the intraoral pressure, volume flow rate, and mass flow rate as a function of time are shown in FIG. 8.

The controller evaluates cycles of pressures and flow rates for internal consistency by evaluating reproducibility for a sequence of breaths. The controller also checks waveforms for internal and mutual consistency with physical laws such as conservation of mass, which requires that the integral of the mass flow rate equal zero over intervals that include an equal number of inhalation and exhalation cycles. Other examples of internal consistency checks that are evaluated by the controller include verification that the mass flow rate is zero when the intraoral pressure is zero, that the mass flow rate is maximal when the pressure is extremal, and conformity with other boundary conditions familiar to those practiced in the art of physics and gas dynamics.

The controller analyzes the pulmonary waveforms in real time to establish the precision of the average waveform, its range of variation, and other statistical measures of the acquired data. The statistical properties of the pulmonary waveforms are used to prompt the client via audible, visual, or written cues to alter their performance while it is being measured, for example to cough, exhale sharply, inhale deeply, hyperventilate, or to perform in other ways behaviors that are familiar to those practiced in the art of respiratory therapy.

The volume of each breath is accurately computed by the controller using the transducers' results for mass flow along with auxiliary data on atmospheric pressure, relative humidity, and temperature, which are acquired either by additional sensors embedded in the transducer or using the geographical coordinates of the client and weather data from the world-wide-web.

According to the present invention, the controller performs these analyses and prompts the user to continue or alter his or her performance until consistent and statistically robust pulmonary diagnostic figures of merit are computed by the controller. Clinically useful diagnostic values include, without limitation, tidal volume, inspiratory and expiratory reserve volumes, residual volume, vital capacity, functional residual capacity, inspiratory capacity, and pulmonary compliance, and other quantities familiar to those practiced in the art of respiratory therapy and described, e.g., in J. A. Seikel, D. W. King, and D. G. Drumright, (loc. Cit.), and A. C. Guyton and J. E. Hall, (loc. Cit.). For example, the controller may prompt the client to continue inhalation and exhalation until the variance in vital capacity is less than some threshold value. The controller evaluates the averages and variances of these values to provide information on statistical confidence in the reported diagnostic quantities. The controller further enhances the value of the method by cross-indexing the diagnostic data with appropriate survey data from other sources such as weather data, geographical data, and environmental factors recorded by other means and available over the internet or other means. In the case of pulmonary function, the weather conditions (barometric pressure, temperature, relative humidity, pollen counts, etc.) are linked to the measured pulmonary values for each patient, and correlation analyses are performed using records from other patients or clients and earlier times.

The present invention is particularly useful for identifying changes in biological performance in populations of people. For example, the aggregate pulmonary performance of a population might be degraded by an environmental influence such as acute pollution, forest fires, pollen blooms, and the like. The quantitative intercomparison of data from individuals dispersed in space and time at the controller permits epidemiological and public health analyses, warnings, and prophylaxis in the presence of sudden changes to the environment.

This aspect of the invention generates accurate and reproducible pulmonary performance characteristics in groups and correlates other properties of the group (age, asthma diagnosis, altitude, humidity, season, and the like) with pulmonary performance using statistical methods. It also can provide a baseline and periodic performance metrics for athletic training purposes.

Auscultation: Another aspect of the present invention characterizes the performance of the heart and lung systems by making digital audio recordings of sound produced by the beating heart and inflating lungs, a process known to those familiar with medical arts as auscultation. The transducer in one embodiment of this aspect of the invention is a microphone with a horn, which is placed on of a client's chest either by himself or by another person. The controller transmits audible or visible prompts to place the transducer at specified locations on the abdomen and then it analyzes the acoustic signature that is transmitted by the transducer. The controller prompts the user to adjust the location and orientation of the microphone until reproducible sound recordings are generated. The controller then archives the recorded sound and compares it to the same patient's historical sounds and the sounds recorded from different patients using digital signal processing means (e.g. Fourier, maximum entropy, wavelet transforms and the like) as set forth by S. L. Marple, Jr. in Digital Spectral Analysis with Applications (New Jersey:Prentice Hall) 1987, the entire contents of which is incorporated by reference herein. The controller may also digitally filter the recorded sounds and display them as visible waveforms, on a client's computer screen, or audible sounds, through a client's audio speakers. The present method is distinguished over the prior art by permitting a non-expert to record auscultation sounds with high fidelity and statistical stability. The method also has the property of providing a systematic digital recording of auscultation sounds that is statistically independent of the observer; prior art methods of auscultation depend on the auditory acuity and experience of the physician or nurse that is using the stethoscope.

Thus the present invention provides auscultation records that are substantially independent of the auditory acuity of the observer. Whereas the auditory acuity of a human observer varies with age, sex, auditory exposure over time, and other factors, with the present invention a controller whose response is precisely calibrated and a transducer whose response has been similarly quantified a priori are used.

Nevi morphology: Another aspect of the present invention involves recording moles or nevi on the epidermis of clients. In one embodiment, a transducer combines a digital camera with a customized light source whose spectral output enhances the contrast between mole and normal skin tissue. The controller prompts the client to photograph images of moles from predetermined perspectives, magnifications, and illumination conditions. The controller then digitally patches these images together to form a map of mole tissue for the client, similar to a Mercator projection of the globe. The nevi are enumerated and characterized by shape, size, color, and location on the epidermis. The client performs this mole scan at periodic intervals, preferably at least once per year, so that an archived history of the number, size, shape, and location of moles is recorded. The diagnostic value of this approach rests in the detection of changes in the number, size, shape, and/or color of moles on skin. The controller uses robust computational algorithms such as pattern correlation, principal component transformation, and the like to elucidate and report these changes to the client and his physician.

Thus, the present invention establishes a quantitative baseline for the morphology of nevi so that pathological changes may be detected by periodic reevaluation of the nevi map.

Refractive error: Yet another aspect of the method of the present invention is a method of screening for refractive error in clients. In one embodiment, a transducer for refractive error measurement is an autorefractor such as the Welch Allyn Sure-Sight vision screening system (Welch-Allyn, Inc., Skaneateles, N.Y.) The controller prompts the client to look into the autorefractor, triggers acquisition of data, computes the refractive error from the raw transducer data, analyzes the fidelity of the refractive error using statistical methods, and compares the measured refractive error with results from an eye chart that is projected onto a computer screen as an interactive test for the client. The autorefractor differs from transducers described in the inventive aspects described above because it is relatively expensive; however, its use does not require sophisticated training when linked to the controller. According to the present method, this transducer would be shipped on loan to a client to be used for a few days and then returned to the company for use by other clients, thereby spreading the capital cost of the transducer over many clients. The combination of low cost and simple operation of the transducer is maintained in this way according to the present method.

Intraocular pressure: Yet another aspect of the present invention is a method for measuring intraocular pressure (IOP) in the eye. Elevated intraocular pressure is diagnostic of glaucoma, which can threaten a patient's vision. IOP measurement, also called tonometry, can be performed by contact with the cornea, as with an indentation (Schiotz) or aplanation (Goldmann) tonometer, or by non-contact displacement using a puffed jet of air. These tests are presently performed only by trained personnel, as misapplication can result in corneal abrasion or irritation. In addition, it is well known that the baseline IOP varies dramatically among individuals and also with time of day, level of stress, and other factors familiar to those practiced in the art of ophthalmology.

According to one embodiment of this aspect of the invention a non-contact acoustic probe is positioned over the eye as if it were an eyewash cup. Acoustic waves are generated by a piezoelectric element or speaker at the base of the cup. These waves impinge on the eye and are reflected with an intensity that depends on the mechanical compliance of the eyeball at each acoustic frequency, which in turn varies with intraocular pressure. The acoustic waves may be transmitted to the eyeball through air or an aqueous solution of sterile liquid such as eyewash. The transducer collects the reflected waves with a microphone and transmits the reflected waveform to the controller. The damping of acoustic waves at selected frequencies and excitation amplitudes is computed by the controller and mathematically transformed to present the IOP and its variance.

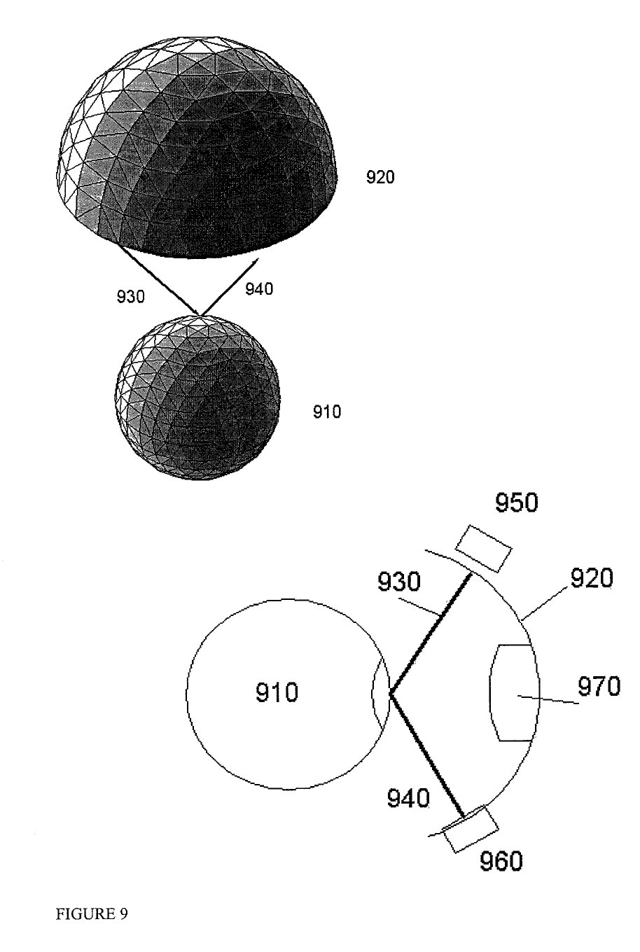

In another embodiment according to this aspect of the invention, the mechanical deflection of the cornea caused by acoustic excitation is detected by a near-infrared optical cantilever, as outlined in FIG. 9.

In yet another embodiment, a sequence of calibrated pressures is applied to the cornea by aliquots of compressed gas delivered through a pulsed valve and an orifice. The deflection of the eyeball is recorded by the acoustic signature of the puff or an optical cantilever.

Thus, intraocular pressure is diagnosed according to the present invention by the following steps:

1. A client places a transducer cup over the left eyeball;

2. The controller triggers application of acoustic or gas dynamic pressure to the cornea;

3. The deflection of the cornea is sensed by optical or acoustic means in the transducer;

4. The raw transducer signal is conveyed over the electromagnetic link to the controller;

5. The controller analyzes the signal and adjusts the operating conditions as required for optimal accuracy and fidelity of the diagnostic measurement; and

6. The measurement is repeated for the right eye.

As in previous aspects of the invention, the controller analyzes multiple measurements under a plurality of calibrated conditions to quantify the accuracy and precision of the diagnostic result. In the absence of adequate accuracy or precision, the measurements are repeated under new conditions to better define the diagnostic result before they are archived and presented to the client by the controller.

This aspect of the invention provides a convenient and accurate measurement of IOP so that a baseline value and excursions therefrom can be established for each client, and correlations between IOP and other biological factors (such as blood glucose concentration) can be quantified.

Audiology: In another aspect of the invention, delocalized diagnostic techniques are used to characterize the auditory frequency response and acuity of a human subject. In one embodiment, the transducer is a set of acoustically insulated and calibrated headphones. The controller transmits a series of sound patterns of varied frequency content and intensity to the headphones while prompting the client to indicate what they hear with a keystroke or pushbutton signal. This series of sounds is adjusted throughout the interview by the controller to ensure accuracy and stability of the measured auditory response.

The preceding description is a representative, but non-limiting illustration of the delocalized biological diagnostic methods according to the present invention. These methods and apparatus provide high accuracy and fidelity diagnostic results without requiring sophisticated laboratory instruments or specially trained operators at the same location as the client. They also improve the convenience of diagnostic evaluation so that intrusive probes or time-consuming appointments are not required to record the temporal evolution of diagnostic data such as blood sugar concentration, pulmonary function, refractive error, intraocular pressure, auditory function, and the like. The above aspects and embodiments are illustrative of the method and must not be construed as limiting other aspects of the method that will be apparent to those practiced in the arts of chemistry, physics, biology, and statistics.

What is claimed is:

1. A method for monitoring a biological property, comprising:
(a) collecting a biological input at a user terminal;
(b) converting the biological input into a first signal in a transducer associated with the user terminal;
(c) transmitting the first signal over a bidirectional link to a controller;
(d) processing the first signal in the controller and generating a second signal;
(e) transmitting the second signal over the bidirectional link to the user terminal; and
(f) converting the second signal into a human-discernible message at the user terminal.

2. A method as recited in claim 1, wherein the biological input comprises a biological specimen.

3. A method as recited in claim 2, wherein the biological specimen is blood.

4. A method as recited in claim 2, wherein the biological specimen is urine.

5. A method as recited in claim 2, wherein the biological specimen is selected from the group consisting of blood, urine, tears, sweat, semen, vaginal swab extract, throat swab extract, sputum, mucous, and breath.

6. A method as recited in claim 1, wherein the biological input comprises a physiological signal, image, or response.

7. A method as recited in claim 6, wherein the physiological signal, image, or response comprises an acoustic signal, a photographic image, a light reflection, a reflected acoustic wave, pressure, an exhalation, or an inhalation.

8. A method as recited in claim 1, wherein the bidirectional link comprises a telephone line, an optical fiber, a cellular phone link, a coaxial cable, a wireless internet link, an infrared data link, a radio frequency link, or a bidirectional satellite pager.

9. A method as recited in claim 1, wherein each of the first and second signals are, independently, an electric signal, a magnetic signal, or an optical signal.

10. A method as recited in claim 1, wherein the user terminal comprises an input port and a user interface.

11. A method as recited in claim 10, wherein the user interface comprises one or more of a computer screen, a key pad, a mouse or other cursor control device, a speaker, and a microphone.

12. A method as recited in claim 10, wherein the user interface comprises a computer screen, a key pad, and a mouse or other curser control device.

13. A method as recited in claim 10, wherein the input port is configured to receive a biological specimen.

14. A method as recited in claim 10, wherein the input port is configured to receive a physiological signal.

15. A method as recited in claim 1, wherein the controller comprises at least one server.

16. A method as recited in claim 1, wherein the human-discernible message comprises an on-screen message, an audio message, or both an on-screen message and an audio message.

17. A method as recited in claim 1, further comprising:
(g) collecting a second biological input at the user terminal;
(h) converting the second biological input into a third signal;
(i) transmitting the third signal over the bidirectional link to the controller;
(j) processing the third signal in the controller and generating a fourth signal;
(k) transmitting the fourth signal over the bidirectional link to the user terminal; and
(l) converting the fourth signal into a human-discernible message at the user terminal.

18. A delocalized apparatus for monitoring a biological property, comprising:
(a) a user terminal comprising an input port and a user interface;
(b) a transducer coupled to the input port;
(c) a bidirectional link coupled to the transducer; and
(d) a controller coupled to the bidirectional link;
wherein, the user terminal and controller have geographically distinct locations;
the transducer is capable of converting a biological input collected at the user terminal into a first signal;
the controller is capable of processing the first signal, generating a second signal, and causing the second signal to be transmitted over the bidirectional link to the user terminal; and
the user terminal is capable of converting the second signal into a human-discernible message.

19. A method for monitoring a biological property in a group of individuals, comprising:
(a) collecting a biological input at each of a plurality of user terminals;
(b) converting each biological input into a first signal in a unique transducer associated with each of the user terminals;
(c) transmitting each first signal over a unique bidirectional link to a controller;
(d) processing all of the first signals in the controller and generating a plurality of second signals;
(e) transmitting a second signal over each unique bidirectional link to each user terminal; and
(f) converting each second signal into a human-discernible message at each user terminal.

20. A method as recited in claim 1, wherein the biological property is blood glucose concentration and the biological input is a blood specimen.

21. A method as recited in claim 1, wherein the biological property is hCG level and the biological input is a blood or urine specimen.

22. A method as recited in claim 1, wherein the biological property is bacteria level and identity and the biological input is a specimen selected from the group consisting of blood, urine, tears, sweat, semen, vaginal swab extract, throat swab extract, sputum, and mucous.

23. A method as recited in claim 1, wherein the biological property is pulmonary function and the biological input is one or more exhalations and/or inhalations.

24. A method as recited in claim 1, wherein the biological property is auscultation and the biological input is an acoustic signal.

25. A method as recited in claim 1, wherein the biological property is nevi morphology and the biological input is a photographic image.

26. A method as recited in claim 1, wherein the biological property is refractive error and the biological input is a light reflection.

27. A method as recited in claim 1, wherein the biological property is intraocular pressure and the biological input is an acoustic or electromagnetic radiation reflection.

28. A method as recited in claim 1, wherein the biological property is auditory response and the biological input is a user's activation of a keypad or cursor control device.
